(19) United States
(12) Patent Application Publication
(10) Pub. No.: US 2021/0319856 A1
(43) Pub. Date: Oct. 14, 2021

(54) METHOD AND APPARATUS FOR IMPROVING BREATHING THERAPY COMPLIANCE

(71) Applicant: Fisher & Paykel Healthcare Limited, Auckland (NZ)

(72) Inventors: Emma Louise Duckworth, Auckland (NZ); Fiona Elizabeth Cresswell, Auckland (NZ); Benjamin Wilson Casse, Auckland (NZ)

(21) Appl. No.: 17/103,608

(22) Filed: Nov. 24, 2020

Related U.S. Application Data
(63) Continuation of application No. 14/406,497, filed on Dec. 8, 2014, now abandoned.
(60) Provisional application No. 61/658,713, filed on Jun. 12, 2012.

(51) Int. Cl. G16H 10/20; G16H 20/30; G16H 50/30

(52) U.S. Cl. CPC G16H 10/20; G16H 50/30; G16H 20/30

(57) ABSTRACT

A method of improving patient adherence to a pressure/flow therapy prescription comprising receiving responses to a questionnaire from a patient, and determining an adherence profile of the patient based on the responses.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### BACKGROUND OF THE INVENTION

### Field of the Invention

The present invention relates to a method and apparatus for improving patient adherence/compliance to pressure therapy or high flow therapy.

### Description of the Related Art

Patients with obstructive sleep apnea (OSA), hypopneas, flow restrictions or other similar disorders can be treated with pressure therapy, for example through CPAP, Bi-PAP or other pressure therapy breathing apparatus. Patients with chronic lung disorders or similar can be treated with high flow therapy and/or supplemental oxygen using a breathing apparatus.

The treatment is provided by way of a pressure therapy apparatus or high flow therapy apparatus, such as a CPAP apparatus or other breathing apparatus that provides pressure/high flow therapy. Generally, the therapy involves providing pressured or high flow gases to a patient through a conduit and interface (e.g. mask) to reduce or eliminate OSA during sleep or treat the lung disorder as appropriate. Patients are prescribed a treatment regime, for example a particular pressure or pressures or particular air flow for a minimum number of hours per night. For a range of reasons, often patients do not adhere to/comply with the treatment prescription. This may be due to apathy, ignorance, lack of comfort or many other reasons.

### SUMMARY OF THE INVENTION

It is an object of the present invention to assist patients to improve their adherence to a pressure/flow therapy prescription.

In one aspect the present invention may be said to consist in a method of generating a questionnaire for use in determining a communication regime for a pressure or flow therapy patient comprising selecting two or more questions associated with two or more of demographics and the following health psychology theories: illness representations theory, social support theory, social cognitive theory, planned behaviour theory.

In another aspect the present invention may be said to consist in a questionnaire for use in determining a communication regime for a pressure or flow therapy patient comprising the selection of two or more questions associated with two or more of demographics and the following health psychology theories: illness representations theory, social support theory, social cognitive theory, planned behaviour theory.

In another aspect the present invention may be said to consist in a system for generating and/or providing a questionnaire to a patient, the questionnaire comprising the selection of two or more questions associated with two or more of demographics and the following health psychology theories: illness representations theory, social support theory, social cognitive theory, planned behaviour theory.

Preferably, the questionnaire comprises:
at least three questions associated with illness representations theory
at least two question from social support theory
at least one question from social cognitive theory (self-efficacy) theory
at least six questions from demographics.

In another aspect the present invention may be said to consist in a method of improving patient adherence to a pressure/flow therapy prescription comprising: receiving responses to a questionnaire from a patient, determining an adherence profile of the patient based on the responses.

Preferably the method further comprises determining a communication regime for the patient based on the adherence profile.

Preferably the method further comprises providing communications to the patient in accordance with the communication regime to improve adherence.

The questionnaire could comprise a selection of two or more questions associated with two or more of demographics and the following health psychology theories: illness representations theory, social support theory, social cognitive theory, planned behaviour theory.

A method of determining the likelihood of a patient adhering to a pressure/flow therapy prescription comprising: receiving responses from a patient to questionnaire, and determining an adherence profile of the patient based on the responses, wherein the questionnaire comprises two or more questions associated with two or more of demographics and the following health psychology theories: illness representations theory, social support theory, social cognitive theory.

Preferably the method further comprises determining a communication regime for the patient based on the adherence profile.

Preferably the method further comprises providing communications to the patient in accordance with the communication regime to improve adherence.

A system for determining a communication regime for a patient to improve their adherence to a pressure or flow therapy prescription comprising:

a database with a questionnaire;

a computer system for:

providing a questionnaire from the database to a patient communications device,

receiving responses from the communications device, and

determining an adherence profile of the patient based on the response.

Preferably, the computer system is also for determining a communication regime for the patient based on the adherence profile.

Preferably the computer system further provides communications to the patient in accordance with the communication regime to improve adherence.

The questionnaire could comprise a selection of two or more questions associated with two or more of demographics and the following health psychology theories: illness representations theory, social support theory, social cognitive theory, planned behaviour theory.

Preferably the communication regime comprises, the frequency/timing, mode and/or content of messaging.

Preferably, the messaging mode can relate to the technology used for messaging and/or the mode of messaging used on that technology.

For example, messaging modes can comprise one or more of the following:
Text message
Instant message
Email
Voice call/messaging
Web browser
App or application
Mail
In person
hardcopy

For example, messaging modes can comprise one or more of the following:
Mobile telephone
Computer
Personal digital assistant (PDA)
Landline telephone
Web enabled device

The terms “adherence” and “compliance” and their derivatives can be used interchangeably. The term “adherence” will generally be used through this specification without any loss of generality.

In this specification where reference has been made to patent specifications, other external documents, or other sources of information, this is generally for the purpose of providing a context for discussing the features of the invention. Unless specifically stated otherwise, reference to such external documents or such sources of information is not to be construed as an admission that such documents or such sources of information, in any jurisdiction, are prior art or form part of the common general knowledge in the art.

The term “comprising” as used in this specification means “consisting at least in part of”. When interpreting each statement in this specification that includes the term “comprising”, features other than that or those prefaced by the term may also be present. Related terms such as “comprise” and “comprises” are to be interpreted in the same manner.

To those skilled in the art to which the invention relates, many changes in construction and widely differing embodiments and applications of the invention will suggest themselves without departing from the scope of the invention as defined in the appended claims. The disclosures and the descriptions herein are purely illustrative and are not intended to be in any sense limiting.

Where specific integers are mentioned herein which have known equivalents in the art to which this invention relates, such known equivalents are deemed to be incorporated herein as if individually set forth.

The invention consists in the foregoing and also envisages constructions of which the following gives examples only.

### BRIEF DESCRIPTION OF THE DRAWINGS

The present invention will be described with reference to the following drawings, of which:

FIG. 1 shows a system that receives input and feedback from patients and uses this to communicate with patients to improve adherence to pressure/flow therapy.

FIG. 2 shows a flow diagram of a method for receiving input/feedback from patients to determine and implement a regime to communicate with patients to improve adherence.

FIG. 3 shows a method of generating questionnaires from the method of FIG. 1.

FIG. 4 shows a method for determining an estimation of adherence risk based on responses to the questionnaires.

FIG. 5 shows a flow diagram of a communication regime made in accordance with the risk profile.

### DETAILED DESCRIPTION OF THE PREFERRED EMBODIMENT

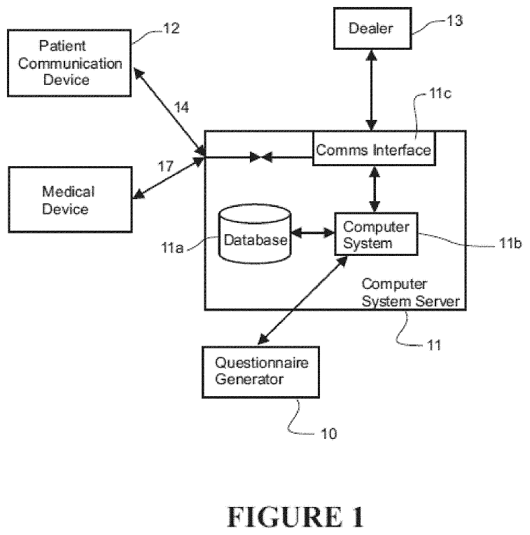

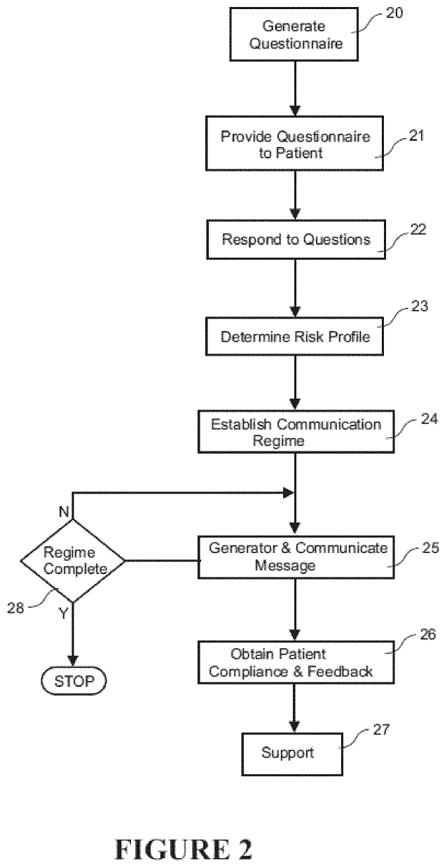

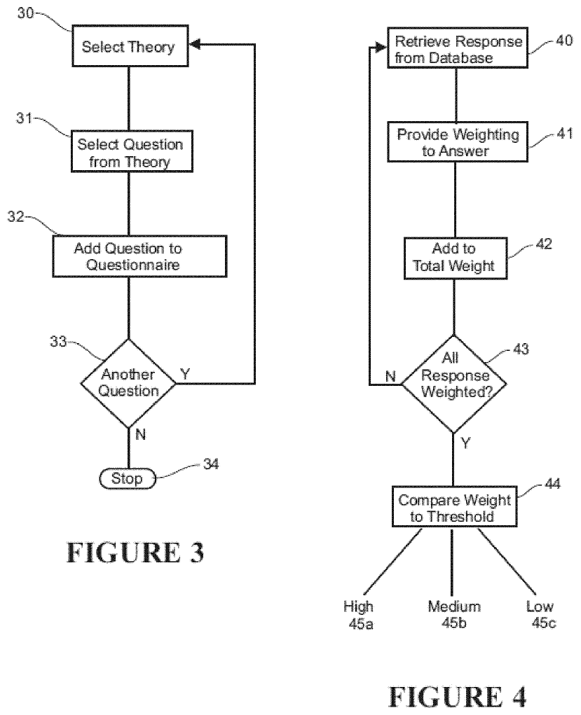

The invention will be described in overview with reference to FIGS. 1 and 2 and an exemplary embodiment (not limiting) will be described with reference to FIGS. 3 to 5.

### Overview

The present inventors have determined that certain questions provided to patients invoke responses that are good predictors of their likelihood of adhering to a pressure/flow therapy prescription. By providing a questionnaire containing such questions, and processing the answers, the present inventors have devised a way to assess the likelihood of a patient adhering to a pressure/flow therapy prescription. This provides an adherence risk profile. Moreover, upon determining that likelihood, the present inventors have further devised regimes for communicating with patients based on their adherence risk profile (“adherence profile”) and associated messages that improve the likelihood that the patient will adhere to a particular pressure/flow therapy prescription.

In general terms, a questionnaire is provided to a patient and they provide their responses. The responses (also termed “answers”) provide information which can lead to good prediction of the likelihood of adhering to a pressure/flow therapy prescription. A (non-) adherence risk profile for the patient is then determined from the responses. The profile could be, for example, the patient being categorised into a (non-)adherence risk category based on their responses. Based on the adherence risk profile, messages and other communications can be relayed to the patient in accordance with a regime. The regime could comprise a frequency and/or mode of communication and optionally the content. The frequency, mode and content of communications assist patients to maintain adherence with their prescription. Subsequent feedback from the breathing apparatus machine and/or other sources can be used to monitor adherence and can be used to alter the regime and/or the patient adherence risk profile. Additional support can also be provided.

FIGS. 1 and 2 show a system and method of operating the system respectively for generating questionnaires, receiving responses to questionnaires from patients, using those responses to determine a communication regime for a patient to improve their adherence to a pressure/flow treatment/therapy prescription and communicating with the patient in accordance with the regime.

First, a questionnaire (also termed a “patient perception questionnaire” or “PPQ”) is generated, step 20. This can be generated by a computer and/or manually by a person or team of persons 10. The questionnaire comprises questions, the responses to which have been determined as being useful alone or in combination for predicting adherence by a patient to a pressure/flow therapy prescription. The questions can be selected from one or more questions that are associated with Demographics and one or more of the following health psychology theories:
Illness representation theory
Social support theory
Social cognitive theory (self-efficacy)
Theory of planned behaviour

Preferably, at least one question comes from Demographics. Illness representations are a part of Leventhal's Self-Regulation theory (Leventhal, H. (1970). Findings and theory in the study of fear communications. In Advances in experimental social psychology (Vol. 5, pp. 119-186). doi: 10.1016/S0065-2601(08)60091-X) which proposes that a person's beliefs and expectations about their illness (illness representations) shape the way a person sees their illness situation and their subsequent health behaviour. This model of health behaviour (Leventhal, H., Meyer, D., & Nerenz, D. (1980). The common sense model of illness danger. In S. Kachman (Ed.), Contributions to medical psychology (Vol. 2, pp. 7-30). New York: Oxford: Pergamon Press.) has been described in a large body of literature, as well many predictive cross-sectional studies being published.

The PPQ items on illness representations dimensions are adapted from: Brief illness perception questionnaire (B-IPQ) (Broadbent, E., Petrie, K. J., Main, J., & Weinman, J. (2006). The brief illness perception questionnaire. Journal of Psychosomatic Research, 60, 631-637. doi: 10.1016/j.jpsychores.2005.10.020). Beliefs about medicine questionnaire (BMQ) (Home, R., Weinman, J., & Hankins, M. (1999). The beliefs about medicine questoinnaire: the development and evaluation of a new method for assessing the congnitive representation of medication. Psychology & Health, 14, 1-24.)

Social support is defined as information from others that one is cared for, loved and valued and part of a network of mutual obligations and communications (Taylor, S. (2012). Health psychology (8 ed.). New York: McGraw-Hill.) Social support has been shown to be beneficial for many aspects of disease including less reported pain, reduced risk of stroke, being less likely to show age-related cognitive decline and showing better adjustment to coronary artery disease (Taylor, S. (2012). Health psychology (8 ed.). New York: McGraw-Hill.)

PPQ questions associated with or taken from Social Support theory ask questions relating to the actual or perceived support (emotional or practical) available to them (the patient) from others including partner, spouse, family, friends, healthcare providers and others they interact with.

Self-efficacy is the centre of Bandura's social cognitive learning theory. This construct first described by Albert Bandura refers to a person's belief that they can succeed at something (Bandura, A. (1977). Self-efficacy: toward a unifying theory of behavioral change. Psychological Review, 84, 191-215.)

The PPQ questions associated with or taken from Self-efficacy (from social-cognitive theory) encompass questions that ask about the patient's feeling of their own ability to complete a task. Any question that asks about their confidence in their ability to complete the task may be determined as a question assessing self-efficacy.

Another psychological model to be considered is the theory of planned behaviour (TPB). This model originally described by Ajzen (Ajzen, I. (1991). The theory of planned behaviour. Organizational behavioural human decision processes, 50, 179-211.) states that an individual's behavioural intention and perceived behavioural control are what influence their behaviour (McEachan, R. R. C., Conner, M., N., T., & Lawton, R. J. (2011). Propspective prediction of health-related behaviors with the theory of planned behaviour: A meta-analysis. Health Psychology Review, 5, 97-144. doi: 10.1080/08870446.2011.613995). This model elicits that there are three types of beliefs that influence behaviour and intention (Farmer, A., Kinmonth, A. L., & Sutton, S. (2006). Measuring beliefs about taking hypoglycaemic medication among people with Type 2 diabetes. Diabetic Medicine, 23, 265-270. doi: 10.1111/j.1464-5491.2005.01778.x). These are behavioural beliefs, which tend to be about personal advantages and disadvantages (e.g., using my CPAP every night will make me feel good), control beliefs, which are about how easy it is to carry out treatment (e.g., having to use my CPAP with my lifestyle will be difficult) and normative beliefs, which are how spouses or partner may view treatment (e.g., my wife thinks using CPAP is a good idea). The theory of planned behaviour can be used to assess intention to use a certain medical treatment.

Demographics describe characteristics of a person or population. This may include but not be limited to age, race, gender, education, employment status, income, marital status, body mass index. The PPQ questions associated with or taken from demographics could relate to any of these.

As an example, the questionnaire comprises:
at least three questions associated with illness representations theory
at least two question from social support theory
at least one question from social cognitive theory (self-efficacy) theory
at least six questions from demographics

Examples of the general nature of questions that can be associated with the above theories and Demographics and selected for the questionnaire are as follows (note, specific questions can be generated from these general nature questions):

Illness representation theory
Does your disease affect your life?
Do you think you have a chronic illness?
Do you understand your disease and treatment?
How concerned are you about using a CPAP?
How much do you think a CPAP can help your OSA?
How well do you understand the benefits of CPAP use?
How long do you think your OSA will continue?
How necessary is a CPAP in controlling your OSA?
How well do you understand the benefits of CPAP use?
How long do you think your OSA will continue?
How necessary is a CPAP in controlling your OSA?

Social support theory
Do you have a spouse/partner?
How supportive is your partner?
Does your partner have a positive attitude?
Social cognitive theory (self-efficacy)
Are you confident in using your treatment?

Theory of planned behaviour
How often do you intend to use a CPAP?

Demographics
What gender are you?
What is your income?
What is your ethnic group?
What is your level of education
What is your marital status?
What is your employment status?

Questions not related to patient perceptions and are not necessarily predictive of adherence but provide useful feedback for other matters such as helping determining types and modes of messaging can also be included. Examples of questions asked about the technology the patient has available are:
Do you have a cell phone? If yes, is it a smart phone?
Do you internet access at home?
Is there good cell-phone coverage in your home?
Other questions can include:
How was your titration experience? Overall, how did you rate you diagnosis and/or titration experience?

These questions are not exhaustive of those that can be used. Further it will be appreciated that additional questions from other sources/theories could also be utilised. Not all questions in the questionnaire have to be predictive of adherence and other questions may be selected for other reasons.

The questions that make up the questionnaire and/or the questionnaire itself can be provided to a database 11a associated with a computer system 11b (together forming a server 11) shown in the system of FIG. 2. This can be operated by or on behalf of a medical equipment dealer/provider, healthcare professional, service provider, insurance provider or other suitable entity. This may or may not be the same entity that generates the questionnaire. The questionnaire is usually generated once in advance of use, although this is not essential. The questions/questionnaire can be generated in advance or in real time.

Next, the method involves querying the patient who is to use the pressure/flow support apparatus 16 using the questionnaire, step 21. The computer system/management server 11b retrieves the questions or questionnaire (hereinafter: questionnaire) from the database 11a and delivers it to a patient communications device 12 through a communications interface 11c via a suitable communications channel 14. The patient communications device 12 could be, for example, a computer, a smart phone (mobile telephone), PDA, landline telephone, web enabled device, or any other device with which the server 11 can communicate with. The questionnaire can be communicated over any suitable communications network either wired or wireless, such as a mobile telephone communication network, or WAN such as the internet, using any suitable mode such as text message, email, web browser, instant messaging, app, widget or application, audio messaging (e.g. voice call/voicemail) or the like. The technology used and/or the manner in which it is used to deliver a message can be termed the “mode”. The questions of the questionnaire are then rendered on the user interface of the patient communications device, such as a screen or by an audio output. While an electronic provision of the questionnaire is most likely, there is no reason why it could not be provided by hardcopy via mail or similar, delivered by human over a telephone or even provided in person at a dealership or similar. The invention is not restricted to the mode of delivery nor the mode of response.

The patient then responds to the questions in the questionnaire, step 22, using any suitable input on the patient communications device such as a keyboard or voice input (or even by mailing back answers or providing them in person or over a telephone). The responses are transmitted over the communications channel 14 back to the server 11 via the interface 11c where they are stored in the database 11a, ready for processing.

Next, the computer system 11b processes the responses from the patient to determine the (non-)adherence risk profile of the patient, step 23. This is the profile that indicates how likely the patient is to adhere to a pressure/flow therapy prescription. The risk profile, for example, could comprise categories of risk, such as high risk, moderate risk and low risk of non-adherence. A scale could be used instead. The invention is not limited to the nature of the risk profile. To determine the risk profile of a patient, the computer system 11b takes the response from each question and provides a weighting. The weighting is based on the strength of the response to a particular question in predicting adherence.

Once the weighting of the response for all questions has been determined and collated, a final weighting is determined which can be used to determine the adherence risk profile, for example by classifying the patient into a risk category. For example, a patient weighting above a certain threshold would be considered high risk of non-adherence, a patient weighting below a certain threshold would be considered a low risk of non-adherence, and a patient weighting between the two could be considered a moderate risk of non-adherence.

This information could be communicated to a medical equipment dealer/provider, healthcare professional, insurance company, service provider or other suitable entity 13.

Next, the computer system/management server 11b establishes a communication regime for the patient based on their risk profile, step 24. The communication regime is matched to the risk profile and has been determined to improve adherence for a patient falling within that risk profile. A communication regime could comprise both the mode and frequency of communication to a patient. For example, the frequency indicates how often (or when) a patient is communicated with, and the mode is by what technological (or even non-technological) mode they are communicated with, such as mobile/landline telephone, computer, PDA via e-mail, app or application, text message, voice message, instant message, web browser, in person, hardcopy or the like. The communication regime can also comprise the content of the messaging, although this is not essential. The content of the messaging can also be determined independently form the communication regime/risk profile.

Once the communication regime has been established, the computer system 11b can communicate with the patient via the communications interface 11c and communications channel 14 in accordance with the regime, step 25. The computer system 11b that provides the communications can be the same that determines the regime (as per FIG. 1), or it can be a separate computer system. The computer system(s) 11b can be operated by or on behalf or the same or different entities. The communications can be operated by/provided on or behalf of a medical equipment dealer, insurance company, service provider, healthcare professional or other interested entity. The computer system 11b generates and provides messages to the patient at the particular times or frequencies as specified by the communication regime. The messages are provided by the mode of the communication regime. The content of the messaging can be taken from a database 11c. The content may be determined based on the responses to the questionnaire and/or some can be simply standard messages or messages determined in some other way. The messages might comprise assistance, motivation, information, encouragement or request feedback. Providing messaging in this way that targets the patient based on their risk profile is more likely to improve their adherence to a prescription regime. The messages continue indefinitely or until the communication regime indicates that messaging is no longer required, step 28.

As a further option, feedback on patient adherence can be obtained at the server 11 from their medical apparatus/pressure/flow therapy apparatus 16 via the communications channel 17/communications interface 11c and this can be used to alter communications or provide communications that complement the communications regime, step 26. For example, if adherence as recorded by the pressure/flow therapy apparatus is below a certain threshold, additional messaging or content may be provided and a different mode might be used. Alternatively or additionally, the adherence risk profile (e.g. the risk category) of the patient could be changed based on the adherence feedback.

As an additional option, the system and method may provide additional support to the patient by way of an interactive programme on a website or pressure/flow therapy apparatus 16 or via the patient communications apparatus 12, step 27. Such support can be in the form of feedback, assistance, motivation, information, encouragement or the like.

The questions, weightings and communication regime that work well to predict and improve adherence have been determined from a clinical trial. A combination of questions from different health psychology theories in combination with demographic variables have been determined to be good predictors of adherence based on the clinical trial.

Ten health psychology questions were used in the research as follows. Note that in this table “self-regulatory model” is mentioned. Illness representations theory is a part of the self-regulatory model. Demographic variables predictive of 90-day adherence are shown underneath, along with Questionnaire variables predictive of 90-day adherence.

Questions Used in the Research

| | Health Psychology | Source of |
|---|---|---|
| Questions | theorem | Question |
| Q1: How much does your OSA affect your | Self-regulatory model | B-IPQ - |
| life? | | consequences item* |
| Q2: How long do you think your OSA will | Self-regulatory model | B-IPQ - time-line |
| continue? | | item* |
| Q3: How necessary is a CPAP in controlling | Self-regulatory model | BMQ - specific |
| your OSA? | | necessity item* |
| Q4: How often do you intend to use a CPAP? | Theory of planned | Formulated for this |
| | behaviour (intention) | research |
| Q5: Do you think your spouse/partner will be | Social support | Formulated for this |
| supportive of you managing your OSA? | | research |
| Q6: How well do you understand the benefits | Self-regulatory model | B-IPQ - coherence |
| of CPAP use? | | item* |
| Q7: How concerned are you about using a | Self-regulatory model | BMQ - specific |
| CPAP? | | concerns item* |
| Q8: How confident are you in using a CPAP | Social cognitive | Formulated for this |
| as instructed? | theory (self-efficacy) | research |
| Q9: How much do you think CPAP can help | Self-regulatory model | B-IPQ - |
| your OSA? | | cure/control item* |
| Q10: My spouse/partners attitude to me using | Social support | Formulated for this |
| CPAP is: positive/negative | | research |

*Adapted for CPAP use

Summary of Associations Between Demographic Variables and 90-Day Adherence

| | Continuous | Adherence status |
|---|---|---|
| | use at | (adherent vs |
| Variable | 90-days | non-adherent) |
| CONTINUOUS | Correlation (ρ) | r |
| BMI | .04 | .00 |
| BLAHI | .14 | .10 |
| TxAHI | .01 | .01 |
| CPAP | .08 | .08 |
| Age | .06 | .03 |
| Cigarettes | .20 | .12 |
| Alcohol | .17* | .19 |
| HCVisits | .08 | .15 |
| DICHOTOMOUS | R | phi |
| PSGSplit | .12 | .09 |
| Comorbs3 | .06 | .10 |
| Sex | .13 | .16 |
| Race | .19* | .18* |
| Depression | .14 | .19* |
| Anxiety | .05 | .05 |
| Residence | .06 | .08 |
| Marital status | .23** | .23** |
| Income | .24** | .30** |
| Employment status | .08 | .13* |

*indicates p < .05,
**indicates p < .01
Note:
As per convention all effect sizes are expressed as positive.
Overview of effect sizes between PPQ baseline and 90-day adherence

| | Continuous | Adherence status |
|---|---|---|
| | use at | (adherent vs |
| | 90-days | non-adherent) |
| Baseline PPQ item | ρ | r |
| Q1 (How much does OSA affect your life) | .01 | .04 |
| Q2 (How long will OSA continue) | .09 | .14 |
| Q3 (How necessary is CPAP in controlling OSA) | .10 | .10 |
| Q4 (How often do you intend to use CPAP) | .19* | .13 |
| Q5 (How supportive is your partner) | .23* | .25** |
| Q6 (Understand benefits of CPAP) | .05 | .01 |
| Q7 (Concerns about CPAP) | .26** | .29*** |
| Q8 (Confident in using CPAP) | .15 | .16* |
| Q9 (How much CPAP can help your OSA) | .20** | .12 |
| Q10 (Spouse/partner's attitude) | .17 | .19* |

*p < .05,
**p < .01,
***p < .001
Note:
As per convention all effect sizes are expressed as positive.

The following table shows for a patient sample what risk profile each patient was categorised into after completing the questionnaire, and what their subsequent adherence was (prior to being communicated with in line with the regime above).

| | No. allocated to group | Adherent | % Adherent |
|---|---|---|---|
| High risk | 24 | 5 | 20.83% |
| Medium risk | 92 | 55 | 59.78% |
| Low | 45 | 43 | 95.56% |

The aim of the clinical study was to test the ability of the PPQ, when delivered at baseline, to predict patient adherence to CPAP at 90-days. The study also aimed to assess changes in PPQ answers over time, assess relationships between the PPQ and adherence at other time-points and to assess subjective adherence and side-effects. A prospective longitudinal study design was employed. A total of 217 patients newly diagnosed with OSA were recruited by Clayton Sleep Institute in St Louis, Mo., USA. Eleven patients were withdrawn and 39 were lost to follow-up because they did not complete the primary endpoint of an objective adherence measurement at 90-days. The final analysis included 167 patients who completed questionnaires at baseline, followed by questionnaires and objective adherence measurements at 14, 60 and 90-days.

The demographic variables that were consistently associated with adherence were race, income and marriage status. When administered at baseline the PPQ questions of higher perceived treatment control, perceived partner support and attitude, self-efficacy and intention to adhere as well as lower concerns about treatment were associated with both 14 and 90-day adherence. When combined in a regression model, demographic and questionnaire variables were able to explain 19.9 and 18.4% of the variance in CPAP adherence at 14 and 90-days respectively. The PPQ items of illness timeline and coherence and self-efficacy increased over time. Objective adherence decreased over time, and was on average over-reported by patients by 42 minutes. Patient reported side-effects significantly increased over time and were associated with 60 and 90-day adherence.

This study shows ability of the PPQ question responses to predict CPAP adherence in OSA or other patients. This research was conducted assessing the ten questions involving health psychology described above as well as demographic variables. Some questions were found to be individually predictive as described above. Once combined together there were some different questions that contributed to the model of adherence prediction. The individual questions stated in the clinical trial and those used in the adherence risk estimator are not necessarily the same.

### Possible Embodiment

A possible embodiment of the invention is described with reference to FIGS. 3 to 5. This embodiment expands on the general embodiment described with reference to FIGS. 1 and 2. The possible embodiment is not limiting and is exemplary only.

Referring to FIG. 3 (which expands on step 20 of FIG. 1) a method of generating a questionnaire is shown. As mentioned previously, the questionnaire can be generated by a computer or a team of persons. The questionnaire is produced by selecting questions that are associated with various patient theories. The questions are those which have been clinically determined to be good indicators of patient adherence/non-adherence and are combined from Demographics and two or more patient health psychology theories.

First, step 30, a theory is selected. Then, step 31, a question from that theory is selected which has a response that provides a good predictor of adherence. The question is then added to the questionnaire, step 32. If the questionnaire is complete the process stops, step 34, or if more questions are to be added, steps 30-32 are repeated, step 33.

In this embodiment, the questionnaire comprises:
at least three questions associated with illness representations theory
at least two question from social support theory
at least one question from social cognitive theory (self-efficacy) theory
at least six questions from demographics.

It would be clear to those skilled in the art that other combinations of questions from various theories are possible. More questions could be added also. The questions themselves can be categorised as questions of a general nature. The general question can be used, or specifically worded questions can then be derived from the general question. Therefore, the questionnaire can comprise general categories of questions or specifically worded questions based on the general category. The general categories or specific wording can be based on demographics and the patient health psychology theories.

A set of questions forming a questionnaire according to this embodiment is set out below according to the health psychology theories they are associated with. Specific questions are indicated and the general questions (where applicable) they are derived from are shown in italics.

From demographics
What gender are you?
What is your income?
What is your ethnic group?
What is your level of education
What is your marital status?
What is your employment status?

From health psychology

From Illness Representation theory
Does your disease affect your life? How much does OSA affect your life?
Are you concerned about your treatment? How concerned are you about using a CPAP?
How helpful is your treatment? How much do you think a CPAP can help your OSA?
From Social Cognitive (self-efficacy) theory
Are you confident in using your treatment? How confident are you in using a CPAP as instructed?
From Social Support theory
Do you have a spouse/partner? Do you think your spouse/partner will be supportive in helping you manage your disease? How supportive is your spouse/partner in helping you manage your OSA?
My spouse/partner's attitude towards my disease is . . . ? How positive is your spouse/partner's attitude to you using CPAP?

Questions not related to patient perceptions and are not necessarily predictive of adherence but provide useful feedback for other matters such as helping determining types and modes of messaging can also be included. Examples of questions asked about the technology the patient has available are:
Do you have a cell phone? If yes, is it a smart phone?
Do you internet access at home?
Is there good cell-phone coverage in your home?

Other questions can include:
How was your titration experience? Overall, how did you rate you diagnosis and/or titration experience?

Each question can be answered by the patient using their device 12, step 22, by providing a yes/no answer or through providing an alphanumeric response. The answer for example, could be a statement or a rating on a scale of 1-10 or similar indicating how much the questions applies to you or similar. A question could alternatively be a statement that the patient can reply to with an indication (e.g. on a scale of 1-10) of how accurately it applies to them. The answer could also be a number (such as your age or salary).

Referring to FIG. 4, the means for determining an adherence risk profile, step 23, will now be described in further detail. Each response to each question is taken in turn from the database, step 40. The answer is then given a weighting based on how good that particular question is in predicting adherence, step 41. An example might be a positive weighting for a positive answer and a negative weighting for a negative answer. Further, a question the response to which is very good at predicting adherence might provide a higher weighting magnitude (positive or negative), whereas a question has responses that are less likely predict adherence are given a lower magnitude weighting (positive or negative). Of course, each question selected has some utility in providing adherence as that is why it has been selected for the questionnaire (although possibly placeholder questions could be provided for various reasons). Where the questions are not simple yes/no questions rather require an answer from a range, then the weighting could be also affected by the range or specific answer in the range that is given.

Once the weighting for a particular response to a question is determined, it is then added to the total weight and then the next question is processed, step 42. Once all question responses have been processed, step 43, a total weighting is determined. This can be used to determine the risk profile of the patient of non-adherence, step 44.

In this embodiment, the risk profile takes the form of three different categories of non-adherence risk, being a) high risk, b) low risk and c) moderate risk. The total weighting is compared to three thresholds, step 44. For example, patients who score −4 or below are considered high risk of non-adherence, step 45a, patients who score between −3 and +4 are considered moderate risk, step 45b, and patient who score 5 or above are considered low risk, step 45c. It will be clear to those skilled in the art that any other weighting suitable regimes could be used and this is one example only.

In one possible embodiment, weightings are attributed to questions as follows. It will be appreciated this example is exemplary only. The 12 variable codings (weightings) for questions are listed below

Demographic variables:
Employment: (response=Yes/No) Self employed=1, unable/disabled=−1 rest=0.
Income: (response=number)<30k=−1, 30-60k=0, Rest=1.
Race: (response=ethnic group) White=0, rest=−1.
Marital: (response=single, married etc) widowed=0, married=1, rest=−1.
Education: (response=high school, degree, PhD etc.) None/high school/GED=−1, rest=0.
Sex: (response=male/female) Male=1, Female=0.
PPQ items:
How much does OSA affect your life?: (response=1-10) 1,10=−1, 9=0, 2−8=1.
How concerned are you about using a CPAP?: (response=1-10) 8-10=−1, 1−7=0, 0=1.
How confident are you in using a CPAP as instructed?: (response=1-10) 0-8=−1, 9-10=1.
How much do you think a CPAP can help your OSA: (response=1-10) 0-8=−1, 9−10=1
How supportive is your spouse/partner in helping you manage your OSA?: (response=1-10) 0-8=−1, 9−10=1. If the question is N/A then use the Marital coding above.
My spouse/partner's attitude to me using a CPAP is: (response=1-10) 2-10=−1, 0−1=1. If the question is N/A then use the Marital coding above.

Once the risk category of the patient is determined, a communication regime can be established. For high risk patients, the regime is as follows:

Message frequency:
Day 1: send welcome message in afternoon, good-luck text in evening
Day 2: send therapy check message in morning plus one more
Day 3: send two messages
Days 4-7: send one message per day
Week 2: send two messages per week
Weeks 3-4: send one message per week

Technology(mode) used:
Preferred: text message to mobile telephone via GSM or Bluetooth
If poor coverage: voicemail or email back to PC

For moderate risk patients, the regime is as follows:

Message frequency:
Day 1: send welcome message in afternoon, good-luck text in evening
Day 2: send therapy check message in morning plus one more
Day 3: send two messages
Days 4-7: send one message per day
Week 2: send two messages per week
Weeks 3-4: send one message per week

Technology(mode) used:
Preferred: text message to mobile telephone via GSM or Bluetooth
If poor coverage: voicemail or email back to PC

For low risk patients, the regime is as follows:

Message frequency:
Day 1: send welcome message in afternoon, good-luck text in evening
Day 2: send a therapy check message morning
Day 4: send one message
Days 7: send one message
Weeks 2-4: send one message per week

Technology used:
Preferred: Voicemail or email back to PC
If no internet access: text message to mobile telephone via GSM or Bluetooth

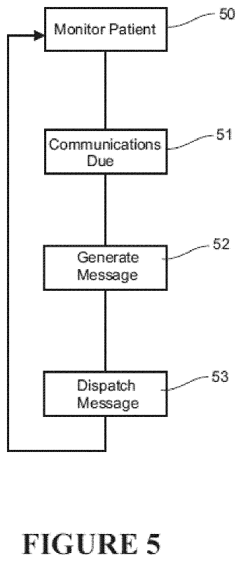

The communication regime is then implemented, step 25, and this is described in more detail with reference to FIG. 5. The patient is monitored, step 50. The computer system 11b is programmed with the communication regime for the particular patient. The particular computer system 11b could have regimes for a large number of patients and manage them all. Referring to the high risk example, communications are made as set out above. The primary mode of delivery is a mobile device (text message is the default, although Smartphone with app capability is possible as well). If patient does not have a mobile device, then voice messages would be sent to their phone. Email messages may also be sent for some patients instead of, or as well as text and voice and applet.

When a communication is due, step 51, the communication computer system 11b generates a message, step 52, at the required time e.g. a welcome message in the afternoon on day 1 and provides that message via the suitable communication mode, step 53 in this case a text message via a GSM network to a mobile telephone. The content for the message is retrieved from a database. Sometimes the content will be a standard message (such as a welcome message) and this is not related in any way to the responses to the questionnaire. However, the content of other messages may be based on the response to the questionnaires. For example, there are message bank areas in the database, the messages in each area relating to particular categories.

Whichever categories the patient has identified as being low in responses to the questionnaires are the categories they will receive messages from at their required time in accordance with the communication regime. If for example a patient's response weighting scored lowly in Illness representations theory questions, then the messages they will receive could preferably come from the Illnesses representations theory bank of messages (as for example set out below). If the weighting was low in another category (such as demographics, social cognitive theory or social support) then the messages can come from that bank of messages. Suitable thresholds for weightings can be set which indicate which messages will be obtained from which categories. The threshold could be based on the relative weightings of answers for each category. Also, messages could be taken from more than one category and delivered together or on a rotated or some other basis. It will be appreciated any other suitable selection regime could be devised.

In one example, each message bank area will have a prioritised list of responses. The patients will receive more important questions first and/or more often. Examples of possible messages in a bank are as per follows:

Welcome and therapy message examples:
Welcome to your adherence program, we look forward to helping you through your CPAP journey
Good-luck with your first night of treatment
How did your first night on treatment go?

Positive experiences message examples:
There is a difference between your home and sleep lab experience, while it may take time to adjust things will be easier using treatment at home
Sometimes it's hard to adjust to CPAP at first, but it will get easier.

Illness representations theory message examples (comprising illness consequences, treatment necessity and treatment cure/control as follows)

Illness consequences examples:
Untreated OSA can seriously affect your daytime functioning with many detrimental effects.
Did you know that untreated OSA can increase your risk of high blood pressure?

Treatment necessity examples:
Increased CPAP usage can help you feel less tired and decrease your risk of chronic conditions such as diabetes
Using your CPAP every day protects you from OSA symptoms.

Treatment cure/control examples:
CPAP can improve your daytime functioning.
Your CPAP controls your OSA by preventing the airway collapse that causes OSA.

Social support questions message examples:
Collaborating with your spouse or partner is a really important part of your CPAP adherence, have you talked to them about your treatment experience today?
Keeping a healthy relationship with your spouse or partner is essential for physical health and well-being, try talking to them about your CPAP experience.

Enhancing self-efficacy message examples (Social cognitive theory):
Sometimes it can be hard to use CPAP at first, but it gets easier the more you use it

Some other message banks will also be available for general areas of encouraging increased CPAP use, such as:

Benefits of routine use examples:
The more you use your CPAP, the better you will feel!

Timeline information (chronicity of disease) examples:
Your OSA is always there even when you don't have symptoms
Your OSA symptoms may come and go but your OSA is always there.

In the questionnaire, some questions are for eliciting responses that will predict adherence while others are used for determining messaging content. For example, questions such as “Overall, how did you rate you diagnosis and/or titration experience?” and the technology questions are for determining message content

Dealer messaging occurs at the start of the program. To become accredited administrators of the method and system described, dealers will be trained so they are proficient in administering the method/system. At time of completion of the PPQ, the algorithm will recommend 2-3 discussion points to the dealer to start the communication and changing of perceptions.

Referring back to FIG. 2, modification of the communications/communication regime can occur based on feedback, step 26. If available (from either text message on mobile phone via GSM/Bluetooth or via email through a PC) objective adherence data will be used to modify the content of messages, the frequency of messages and to offer support from their healthcare provider. This comes from the pressure/flow therapy device 15 which can provide information back to the server 11. Some examples of alterations due to feedback are as follows.

In one example, if a patient is initially categorised as low-risk of non-adherence from the questionnaire responses and if it turns out that usage is <3 hours per night for the first three nights they will be moved to the moderate messaging frequency group (moderate risk of non-adherence) and the communications regime for that patient will changes to that for the moderate risk group.

In another example, for a patent of any risk category, if usage is <3 hours per night for the first 3 nights and/or if f usage is <3 hours per night for the first 7 nights then the following happens. A message is sent from the server 11 to the patient communications device 12 offering help from their healthcare provider. If a “yes” response received then the dealer is notified to contact the patient. If the response is “no”, an encouraging message is sent regarding continued use. The messaging can be adjusted based on usage, such as “You're not yet meeting your adherence goal, try for one hour more tonight.”

In another example, if usage is <3 hours per night for the first 14/21/30 nights, then the following happens. A message is sent from the server 11 offering help from their healthcare provider. A message is sent from the server 11 to the patient communications device 12 offering help from their healthcare provider. If a “yes” response received then the dealer is notified to contact the patient. If the response is “no”, an encouraging message is sent regarding continued use. A message is then sent asking if they would like to continue on the program e.g. “It doesn't look like you're meeting your adherence goal, this may compromise your insurance funding. Would you like our support to continue?” If the response is “yes”, the patient is started again on the “high-risk” path of messaging, starting at day 2. If the response is “no”, a message is sent about getting help from healthcare provider (as described above).

In another example, if usage is <3 hours per night, at various times during the program, a suffix or prefix could be added to the message relating to usage e.g., “We see you have only used your device for 2 hours each night, try extra collaboration with your partner.”

Additional support can also be provided, step 19. Via a CPAP or an app or a website each morning the patient will asked to:
Rate the quality of your sleep
Ask the spouse to rate the quality of their sleep
Combine this with objective adherence data from the machine

Via a CPAP or an app or a website each morning the patient/spouse will be asked to:

### Patient

Rate their energy levels now (1-10)
Rate their mood now (1-10)

### Spouse

Rate patient's energy level (1-10)
Rate patient's mood (1-10)

A Yawn app will ask patient how many times the patient has yawned.

A CPAP coach can be provided for both patient and partner. Persons with similar experiences to help and guide them. The Coach takes the “form” most similar to the patient (or spouse). It offers videos and cartoon

### Ongoing Support:

Algorithm developed that reports on relationship between objective and subjective measures
To promote personal necessity and benefits of treatment
Relationship reported back to patient (text, email, HZ)
Could report on associations with the literature (e.g. at least four hours per night of sleep reduce your risk of heart attack)
Add motivational messages for ongoing support
Graphical representation of relationship over time (HZ and app)
E.g., hours of use versus energy levels
Give consent to dealers/physicians to access
Where a positive relationship is unclear
Have a bank of generic messages
Report on associations in the literature
Consider/include information on other lifestyle factors e.g., diet/exercise
Modules for comorbidities
Hypertension
Diabetes

Modules for lifestyle factors
Stress management
Weight management

Replenishment through Web portal

1. (canceled)

2. A system for assisting one or more patients to adhere to a personal respiratory prescription, the system comprising:
one or more medical devices, wherein each medical device of the one or more medical devices is associated with one of the one or more patients, where each of the one or more medical devices comprise a blower and a controller, wherein the controller is configured to control the blower so that the respective medical device provides personal respiratory therapy to the respective patient according to the personal respiratory prescription;
a computer system comprising a database a processor, and a memory, wherein the database comprises one or more questionnaires configured to be predictive of the respective patient's likelihood of adhering to the personal respiratory prescription, wherein each of the one or more questionnaires comprises one or more questions; and
one or more patient communication devices, wherein each of the one or more patient devices is associated with one of the one or more patients, wherein each of the one or more patient communication devices is configured to wirelessly communicate with the respective medical device and the computer system, wherein each of the one or more patient communication devices comprises a communication interface, a processor, and a screen;
wherein the computer system is configured to:
transmit the respective questionnaire to the respective patient communication device,
receive a response to one or more questions in the respective questionnaire from the respective patient communication device,
process each of the one or more responses at the processor by weighting each of the one or more responses based on a predetermined criteria,
determine a total weighting based on the weighting of each of the one or more responses,
based on the total weighting, determine a respective risk profile for the respective patient, wherein the respective risk profile provides information related to the respective patient's likelihood of adhering to the personal respiratory prescription, and
based on the determined respective risk profile, send one or more messages to the respective patient communication device to remind the respective patient of the personal respiratory prescription.

3. The system of claim 2, wherein respective prescription parameters are stored within a memory of the respective medical device, the controller of the respective medical device being configured to automatically access the memory and control the blower according to the respective prescription parameters.

4. The system of claim 2, wherein the processor of the computer system is configured to provide the respective questionnaire to the respective patient communication device, and wherein the respective patient communication device is configured to transmit the respective questionnaire to the respective medical device.

5. The system of claim 2, wherein the processor of the computer system is configured to directly transmit the respective questionnaire to the respective medical device, and wherein the respective questionnaire is presented on a graphical user interface of the respective medical device.

6. The system of claim 2, wherein the processor of the computer system is configured to determine a respective communication regime for the respective patient based on the respective determined risk profile.

7. The system of claim 6, wherein the processor of the computer system is configured to provide the one or more messages to the respective patient communication device of the respective patient in accordance to the determined respective communication regime for the respective patient.

8. The system of claim 6, wherein the respective communication regime comprises a frequency of communication, a mode of communication, and/or a content of messaging.

9. The system of claim 2, wherein the weighting for each of the one or more responses is based on how well the particular question of the one or more questions is in predicting the respective patient's likelihood of adherence to the personal respiratory prescription.

10. The system of claim 2, wherein the respective risk profiles comprise one of a) a high risk of adherence profile, b) a medium risk of adherence profile, or c) a low risk of adherence profile.

11. The system of claim 10, wherein the respective patient's respective risk profile is automatically determined to be the high risk of adherence profile if the total weighting is above a first threshold.

12. The system of claim 11, wherein the respective patient's respective risk profile is automatically determined to be the low risk of adherence profile if the total weighting is below a second threshold.

13. The system of claim 12, wherein the respective patient's respective risk profile is automatically determined to be the medium risk of adherence profile if the total weighting is between the first and second thresholds.

14. A system for assisting one or more patients to adhere to a personal respiratory prescription, the system comprising:
one or more medical devices, wherein each medical device of the one or more medical devices is associated with one of the one or more patients, where each of the one or more medical devices comprise a blower and a controller, wherein the controller is configured to control the blower so that the respective medical device provides personal respiratory therapy to the respective patient according to the personal respiratory prescription;
a computer system comprising a database a processor, and a memory, wherein the database comprises one or more questionnaires configured to be predictive of the respective patient's likelihood of adhering to the personal respiratory prescription, wherein each of the one or more questionnaires comprises one or more questions; and
one or more patient communication devices, wherein each of the one or more patient devices is associated with one of the one or more patients, wherein each of the one or more patient communication devices is configured to wirelessly communicate with the respective medical device and the computer system, wherein each of the one or more patient communication devices comprises a communication interface, a processor, and a screen;
wherein the computer system is configured to:
transmit the respective questionnaire to the respective medical device,
receive a response to one or more questions in the respective questionnaire from the respective medical device,
process each of the one or more responses at the processor by weighting each of the one or more responses based on a predetermined criteria,
determine a total weighting based on the weighting of each of the one or more responses,
based on the total weighting, determine a respective risk profile for the respective patient, wherein the respective risk profile provides information related to the respective patient's likelihood of adhering to the personal respiratory prescription, and
based on the determined respective risk profile, send one or more messages to the respective medical device to remind the respective patient of the personal respiratory prescription.

15. The system of claim 14, wherein respective prescription parameters are stored within a memory of the respective medical device, the controller of the respective medical device being configured to automatically access the memory and control the blower according to the respective prescription parameters.

16. The system of claim 14, wherein the processor of the computer system is configured to provide the respective questionnaire to the respective patient communication device, and wherein the respective patient communication device is configured to transmit the respective questionnaire to the respective medical device.

17. The system of claim 14, wherein the processor of the computer system is configured to directly transmit the respective questionnaire to the respective medical device, and wherein the respective questionnaire is presented on a graphical user interface of the respective medical device.

18. The system of claim 14, wherein the processor of the computer system is configured to determine a respective communication regime for the respective patient based on the respective determined risk profile.

19. The system of claim 18, wherein the processor of the computer system is configured to provide the one or more messages to the respective patient communication device of the respective patient in accordance to the determined respective communication regime for the respective patient.

20. The system of claim 18, wherein the respective communication regime comprises a frequency of communication, a mode of communication, and/or a content of messaging.

21. The system of claim 14, wherein the weighting for each of the one or more responses is based on how well the particular question of the one or more questions is in predicting the respective patient's likelihood of adherence to the personal respiratory prescription.

22. The system of claim 14, wherein the respective risk profiles comprise one of a) a high risk of adherence profile, b) a medium risk of adherence profile, or c) a low risk of adherence profile.

23. The system of claim 22, wherein the respective patient's respective risk profile is automatically determined to be the high risk of adherence profile if the total weighting is above a first threshold.

24. The system of claim 23, wherein the respective patient's respective risk profile is automatically determined to be the low risk of adherence profile if the total weighting is below a second threshold.

25. The system of claim 24, wherein the respective patient's respective risk profile is automatically determined to be the medium risk of adherence profile if the total weighting is between the first and second thresholds.
